(19) United States
(12) Patent Application Publication
(10) Pub. No.: US 2010/0331927 A1
(43) Pub. Date: Dec. 30, 2010

(54) FEEDBACK-CONTROLLED METHOD FOR DELIVERING PHOTODYNAMIC THERAPY AND RELATED INSTRUMENTATION

(76) Inventors: William J. Cottrell, Dallas, TX (US); Thomas H. Foster, Rochester, NY (US); Allan R. Oseroff, Buffalo, NY (US)

Correspondence Address: BLANK ROME LLP, WATERGATE, 600 NEW HAMPSHIRE AVENUE, N.W., WASHINGTON, DC, 20037, US

(21) Appl. No.: 12/598,409

(22) PCT Filed: May 2, 2008
(86) PCT No.: PCT/US08/62494

§ 371 Date: Sep. 16, 2010

Related U.S. Application Data
(60) Provisional application No. 60/924,173, filed on May 2, 2007.

(51) Int. Cl. A61N 5/06

(52) U.S. Cl. 607/88

(57) ABSTRACT

A method for delivering photodynamic therapy (PDT) while performing dose metric monitoring and treatment feedback-driven control is presented. Photodynamic therapy is initiated with irradiation of light at a first irradiance. A set of fluorescence and reflectance spectroscopic measurements are taken at prescribed intervals during the therapy of the treatment region. Spectra are analyzed to determine dose metrics of the therapy such as fluorescence photobleaching of the sensitizer and blood oxygen status and optical properties of the treatment region. This information is then used to determine an optimal fluence rate given those parameters and the region is irradiated with a second irradiance. This process is continued until either the entire prescribed fluence is delivered to the region or a predetermined extent of photosensitizer bleaching is achieved.

### REFERENCE TO RELATED APPLICATIONS

The present application claims the benefit of U.S. Provisional Patent Application No. 60/924,173, filed May 2, 2007. The following applications disclose related subject matter which can be used in the present invention: WO2006/025940 and U.S. patent application Ser. No. 11/783,199. The disclosures of all of those applications are hereby incorporated by reference in their entireties into the present disclosure.

### FIELD OF THE INVENTION

This invention relates generally to photodynamic therapy (PDT). More specifically, it relates to spectroscopic measurements during PDT which contribute to feedback in the system and changes to PDT delivery.

### DESCRIPTION OF RELATED ART

Over the last several decades, PDT has become an established treatment modality for a variety of medical conditions including actinic keratosis, Barrett's esophagus, acne vulgaris, and most notably, several types of cancer. Specifically, photodynamic therapy using 5-aminolevulinic acid (ALA) is an effective therapy for treating basal cell carcinoma (BCC), with patients demonstrating high lesion clearance and excellent cosmetic outcomes. In this therapy, the prodrug ALA is absorbed into the skin and converted to the photosensitizer protoporphyrin IX (PpIX) in the heme-biosynthetic pathway. PpIX is then excited by red light, leading to molecular energy transfer to oxygen and creation of singlet oxygen, a short lived and highly reactive cytotoxic species whose reactions with sensitive targets lead to cell death. Though photodynamic response occurs in all areas where photosensitizer, light, and oxygen are present, the interaction among these quantities complicates dosimetry, sometimes in counterintuitive ways. For example, as the fluence rate of a treatment is increased, the PDT reaction may be driven faster and more oxygen may be consumed, attenuating therapy. This effect has been verified in BCC patients undergoing Photofrin PDT, where transient, treatment induced tumor hypoxia was observed when treatment was performed with 150 mW cm⁻², while oxygenation was maintained with a fluence rate of 30 mW cm⁻². Blood oxygenation has also been shown to have additional effects on treatment efficacy and dosimetry. For example, blood absorption in the therapeutic window between 585 and 800 nm is dominated by deoxyhemoglobin. Therefore, higher blood oxygenation is associated with less absorption, deeper light penetration, and larger treatment volumes.

Current PDT treatment protocols prescribe four quantities that define the delivered dose: administered photosensitizer concentration, incident fluence, fluence rate, and the drug-light interval. Dose prescriptions using these parameters, however, have proved inconsistent in predicting treatment efficacy. In addition to lesion-to-lesion heterogeneities in both optical properties and lesion histology, limitations in treatment efficacy are governed by the fact that PDT is a dynamic and multivariable process during which oxygen concentrations, photosensitizer concentration, and even the fluence rate within the tissue may vary.

PDT optimization utilizing explicit dose metric monitoring has been employed in the clinical arena only in limited cases, mainly as the result of technical challenges. Measurements of optical fluence and fluence rates during therapy have received recent attention because these parameters may be varied in the treatment and are often accessible. For example, fluence monitoring has been used to help protect healthy tissue in hollow organs during PDT treatments of esophageal cancer and Barrett's esophagus, bladder, and bronchi. It has also been used interstitially where the measurements are direct and therefore most accurate. Tumor oxygenation status has also been explored using polarographic needle electrodes, phosphorescence quenching, and nitroaromatic binding. Several researchers have therefore investigated the use of implicit dosimetry in PDT, in which a surrogate measure of dose, generated further down the photochemical treatment pathway, is made through accessible parameters. Photosensitizer photobleaching is an attractive surrogate dose metric as it is often mediated by mechanisms similar to oxidation of cellular targets, is accessible through non-invasive spectroscopy, and may be correlated with singlet oxygen deposition under many clinically relevant conditions. It has also been shown that the bleaching rate of PpIX is related to oxygen availability and is predictive of therapeutic outcome. Other spectroscopically-accessible parameters such as production of photosensitizer photoproducts and accumulation of endogenous biological fluorophores may offer promise as dose metrics.

Treatment fractionation is a technique used in oxygen dependent radiotherapy that has been adapted for use in PDT. In this treatment modality, the fluence rate is maintained but the light delivery is broken up so that the oxygen tension within the target tissue has time to recover. Irradiation fractionation was used with a VX-2 transplanted skin carcinoma treated with Photofrin while monitoring oxygen tension using transcutaneous oxygen electrodes. This experiment revealed that at 50 mW/cm² the oxygen recovery time is roughly twice the irradiation time. A theoretical model was more recently created which suggests that the vasculature spacing is much more important in determining the proper length of the dark period during delivery fractionation than either the fractionation intervals or the sensitizer concentration. As in the case of fluence rate for delivery, the efficacy of the PDT treatment is dramatically affected through fractionation. For example, a rat bladder tumor model showed that 60s/60s, light interval/dark interval, light fractionation improved phototoxicity 100-fold in an ALA-induced PpIX treatment and 1000-fold in a BPD-MA treatment. A significantly longer dark interval of 75 minutes resulted in a 3-fold increase in tumor volume doubling time for the case of ALA-induced PpIX treatment of subcutaneously transplanted tumors in rats, attributed primarily to metabolically driven resupply of PpIX after initial bleaching effects.

It has been observed that ALA-PDT is painful in a variety of cases, often requiring local anesthesia or conscious sedation. It has also been observed that the pain is significantly relieved when treating at lower fluence rates.

### SUMMARY OF THE INVENTION

It is an object of the invention to overcome the above-noted drawbacks.

To achieve the above and other objects, the present invention is directed to a system and method for delivering photodynamic therapy (PDT) while performing dose metric monitoring and treatment feedback-driven control. Photodynamic therapy is initiated with irradiation of light at a first irradiance. A set of fluorescence and reflectance spectroscopic measurements are taken at prescribed intervals during the therapy of the treatment region. Spectra are analyzed to determine dose metrics of the therapy such as fluorescence photobleaching of the sensitizer and blood oxygen status and optical properties of the treatment region. This information is then used to determine an optimal fluence rate given those parameters and the region is irradiated with a second irradiance. This process is continued until either the entire prescribed fluence is delivered to the region or a predetermined extent of photosensitizer bleaching is achieved.

### BRIEF DESCRIPTION OF THE DRAWINGS

A preferred embodiment will be set forth with reference to the drawings, in which:

FIGS. 1A and 1B show absorption curves for oxyhemoglobin, deoxyhemoglobin and water;

FIGS. 2A-2C show an instrument in which the preferred embodiment can be implemented;

FIGS. 3A-3D show a probe;

FIGS. 4A and 4B show fluorescence spectra measured with the instrument of FIGS. 2A-2C;

FIGS. 5A and 5B show contributions to fluorescence;

FIGS. 6A and 6B show white light reflectance spectra;

FIG. 7 is a flow chart showing the operation of the present invention; and

FIG. 8 shows a time course of therapy for PDT.

### DETAILED DESCRIPTION OF THE PREFERRED EMBODIMENT

A preferred embodiment will now be set forth in detail with reference to the drawings, in which like reference numerals refer to like elements or steps throughout.

### Photobleaching

Singlet oxygen, ¹O₂, generated during PDT can oxidize the photosensitizer (oxygen-mediated photobleaching). Additionally, triplet-state photosensitizer reactions provide a competing bleaching mechanism whereby bleaching is mediated by the triplet-state photosensitizer molecule directly (triplet-state photobleaching). Both mechanisms reduce tissue fluorescence. Fluorescence photobleaching and the rate of photobleaching of both photosensitizer and other fluorophores can therefore be used to implicitly report dose.

Incorporation of photosensitizer photobleaching as a dosimetry parameter requires a careful examination of the photochemical pathway governing the photosensitizer and ¹O₂ dynamics. Photosensitizer photobleaching in vivo is accessible through fluorescence measurements and has been investigated by several research groups for a variety of photosensitizers. Several studies examining the in vivo photobleaching during ALA-PDT have shown correlation between the rate of photobleaching and biological response, though other studies have shown no correlation. Inconsistencies between treatment protocols in treatment wavelengths, corrections for tissue optical properties, and spectral analysis, however, are likely causes of the inconsistencies and are being addressed by this and other research groups. Quantitative information about fluorophore bleaching can be gathered non-invasively in vivo and corrected for the effects of tissue optical properties.

### Tissue Oxygenation Measurements Through Diffuse Reflectance

Oxygen depletion in tissues during PDT, changes to tissue optical properties due to oxygen tension, changes in cell surviving fraction with irradiance, oxygen-dependent changes to prodrug metabolism, and other experimental and theoretical results support oxygen dynamics as playing a major role in PDT dosimetry. Since ¹O₂ is irreversibly consumed upon target oxidation and resupply of molecular oxygen to the tissue through oxygen diffusion is determined by a variety of biological factors, the resulting dynamics of tissue oxygenation are complex and partially governed by the PDT treatment itself. For example, it has been demonstrated that as the fluence rate is increased, the PDT reaction is driven faster and more molecular oxygen is consumed. At higher fluence rates the oxygen consumed by the PDT process outpaces that replaced by diffusion for cells not in close proximity to a capillary, producing hypoxic regions and preventing further dose delivery.

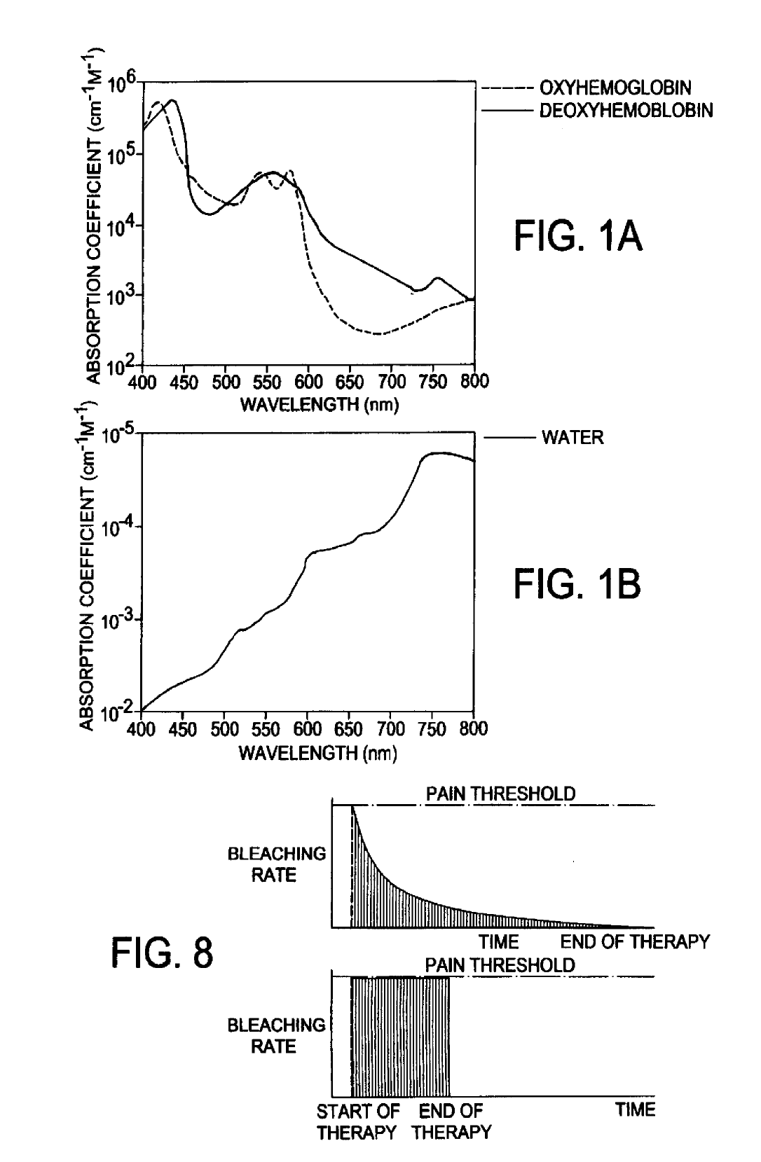

Transcutaneous oxygen electrodes, phosphorescence quenching, and nitroaromatic binding have been explored for oxygen measurement. Two-dimensional mapping of oxygen tension within small tumors has been accomplished with the use of oxygen-quenched metalloporphyrin dyes. Detection of ¹O₂ through its characteristic 1270 nm luminescence has recently been demonstrated in both cells and tissues treated with tetrasulfonated aluminum phtalocyanine. One of the most clinically promising techniques for determining oxygen tension, however, is white light spectroscopy, providing chromophore measurements of the blood. Tissue absorption in the therapeutic window, between 450 nm and 900 nm, is dominated by chromophores oxy- and deoxyhemoglobin and water, the extinction coefficients of which are plotted in FIGS. 1A and 1B.

This allows the absorption in tissue as a function of wavelength to be approximated as

μₐ(λ)=a₁μ_{aHbO}_{₂}(λ)+a₂μ_{aHb}(λ)+a₃μ_{aH}_{₂}_{O}(λ)

where λ_{aHbO}_{₂}(λ), μ_{aHb}(λ) and μ_{aH}_{₂}^{O}(λ) are the millimolar absorption coefficients of HbO₂, Hb and water, respectively, and a₁, a₂, and a₃ represent their respective concentrations. White light diffuse reflectance spectroscopy has been used to accurately determine the oxygenation state of the blood as well as blood volume using a single source-detector separation. Oxygen saturation is found using the Hill equation,

$${SO}_{2} = \frac{[ Hb {O}_{2} ]}{[ {Hb O}_{2} ] + [ Hb ]} = \frac{{( {p O}_{2} )}^{n}}{{( {p O}_{2} )}^{n} + {( {p}_{50} )}^{n}} ,$$

where pO₂ is the oxygen partial pressure, p₅₀ is the partial pressure at which half the heme binding sites are bound to oxygen, and n is the Hill parameter, a measure of the cooperativity of binding. Tissue oxygenation has also been shown to have secondary effects on treatment efficacy and dosimetry. The absorption of tissues being treated with light of a wavelength between 585 nm and 800 nm may be dominated by deoxyhemoglobin. Therefore, better blood oxygenation is associated with less tissue absorption, deeper light penetration, and larger treatment volumes.

### Photoproducts and Bio-Indicators

In addition to fluorescence photobleaching, fluorescent photoproducts offer other implicit measures of dose. In the case of ALA-PDT, for example, two major fluorescent photoproducts have been reported. Photoproduct I has a fluorescence emission maximum at 676 nm, and photoproduct II has an emission maximum near 654 nm. The literature suggests that these fluorescent photoproducts, often called photoprotoporphyrins, are chlorins resulting from the oxidation of the photosensitizer by ¹O₂. The fluorescent photoproducts also exhibit photodynamic action, and spectroscopic studies in vivo have suggested that photoproduct I may bleach through further oxidation into photoproduct II. The dependence of photoproduct formation on the presence of ¹O₂ makes photoproducts attractive for use as dose reporters, though they are not generated with all photosensitizers and under all treatment conditions.

In addition to detection and characterization of photoproducts, spectroscopic techniques can be employed to detect biological signatures of PDT-induced damage. ALA-incubated carcinoma cells have shown fluorescent peaks near 620 nm as have bladder contents of ALA treated rats and normal rat skin treated with ALA-PDT. These have been assigned to the water soluble porphyrins uroporphyrin (up) and coproporphyrin (cp). Cp and up are endogenous porphyrin compounds that are manufactured in the heme synthesis pathway between ALA and PpIX. These compounds are created in the intracellular cytosol just prior to being taken up by the mitochondria where the conversion to PpIX and heme occurs. It has been suggested that the appearance of these water soluble porphyrins indicates mitochondrial damage and that they can be used as a direct reporter of PDT-induced biological damage.

### Instrumentation

The instrument described below is based on the system that has been reduced to practice and was described in the above-cited WO2006/025940. The preferred embodiment extends the capabilities to include real time feedback-driven control of treatment irradiance and fluence.

### System Description and Representative Results

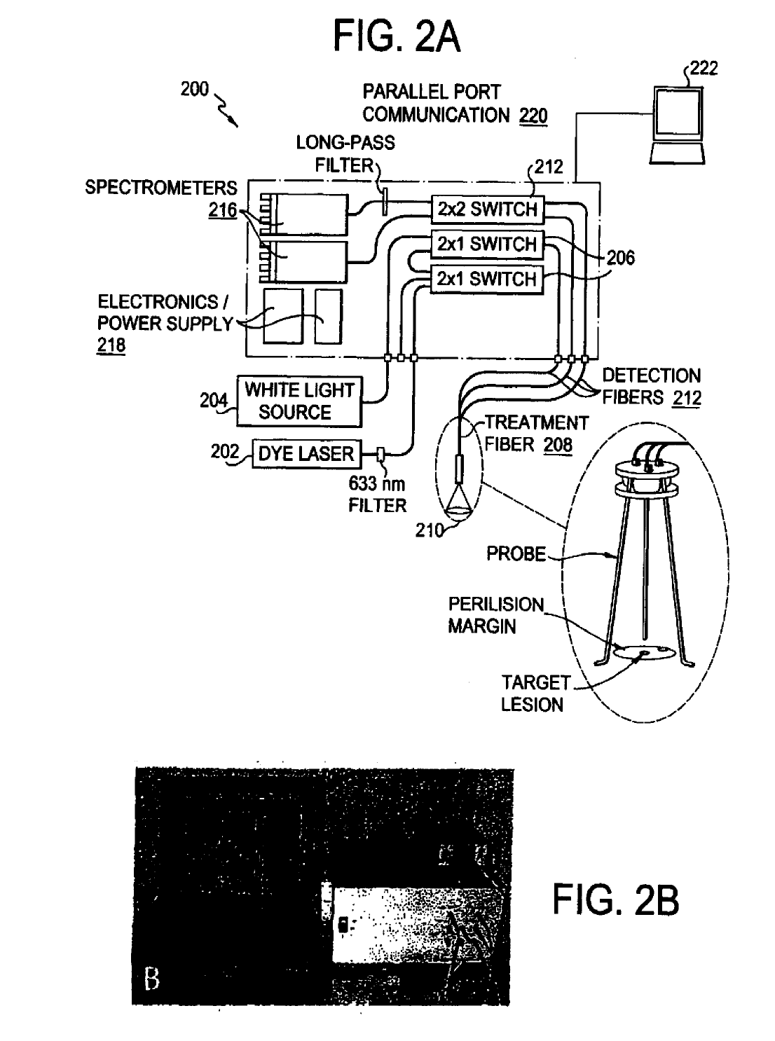

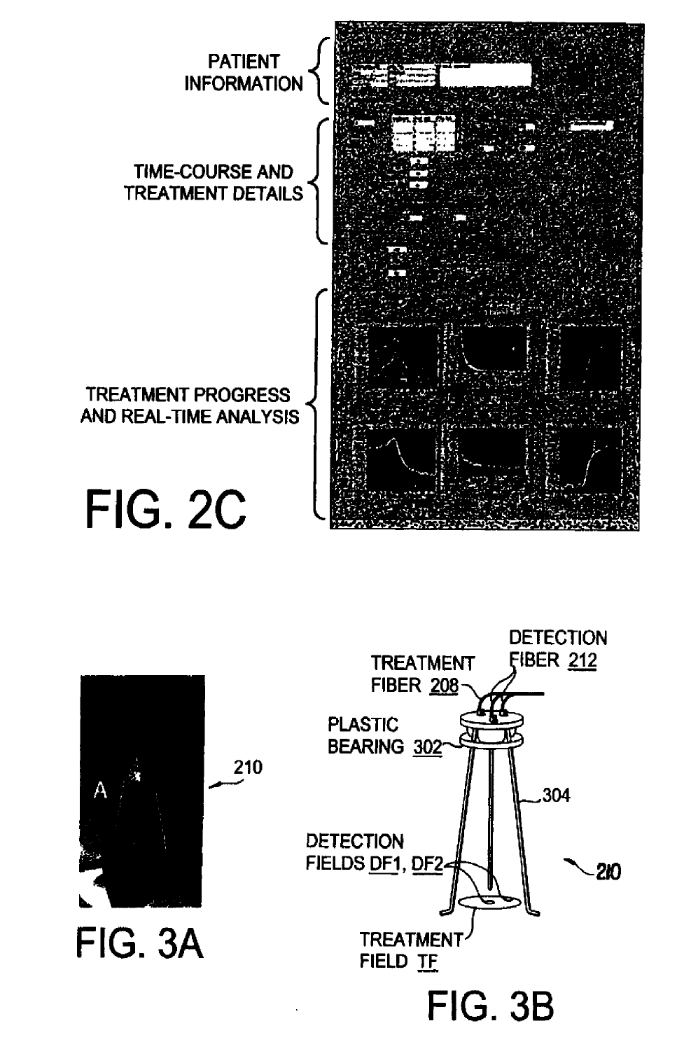

The instrumentation was designed to deliver the PDT treatment beam and perform fluorescence and reflectance spectroscopies during PDT of skin lesions on two spatially-resolved measurement regions. One field is located inside the lesion and the other is located within the adjacent perilesional margin. The instrumentation design was heavily influenced by restrictions imposed by the currently approved clinical treatment protocol at RPCI, which includes delivering the treatment beam through a 600 μm-core fiber terminated with a GRIN lens that is housed in an off-surface probe positioned 80 mm from the skin. The system is shown in FIGS. 2A-2C.

FIGS. 2A and 2B a schematic illustration and a photograph, respectively, of the computer-controlled clinical instrumentation developed for application at Roswell Park Cancer Institute. The instrument 200 includes a dye laser 202, a white light source 20, and two 2×1 opto-mechanical switches 206 that control delivery of the treatment beam and white light to the tissue through a treatment fiber 208 and an off-surface probe 210. Detection fibers 212 and a 2×2 switch 214 are also used to direct subsequent fluorescence and reflectance signals to a pair of thermoelectrically-cooled spectrometers 216. Electronics/power supply 218 and a parallel port communication 220 (or other suitable communication, such as USB or wireless) to a computer 222.

FIG. 2C shows a user interface for instrumentation developed in LabVIEW. The user inputs patient information and the PDT treatment time course, consisting of treatment and acquisition intervals in a spreadsheet format. Acquisition of baseline information such as dark signal and initial reflectance spectra are also initiated by the user. During the treatment, white light reflectance spectra and SVD-analyzed fluorescence spectra are displayed in near real-time to the clinician. Additionally, the program displays the progression of the PpIX bleaching curves from both detection regions as each fluorescence spectrum is analyzed. The two peaks seen in the reflectance spectra of the display near 629 nm and 633 nm are the result of stray light in the clinic due to multiple simultaneous PDT treatments of nearby lesions.

The instrument's optical delivery chain makes use of two light sources: an argon-ion-pumped dye laser (Coherent Inc., Santa Clara, Calif.) operating at 633 nm and an SMA-coupled tungsten-halogen source with internal TTL shutter (Avantes, Boulder, Colo.). The output of the dye laser is filtered with a 632.8 nm laser line filter (Semrock, Rochester, N.Y.) and serves as the PDT treatment beam. Filtering the beam minimizes source bleed-through into the fluorescence detection channel when spectra are acquired. The filtered laser beam is coupled into a first 2×1 fiber-optic switch. The output of the switch and the white light source are then coupled to a second 2×1 switch whose output channel is the treatment fiber, the distal end of which is mounted in a tripod probe with a beam-aiming bearing adjustment. Two lens-terminated detection fibers are also terminated in the probe and coupled into a 2×2 fiber switch. One output channel of this switch is dedicated to the detection of white light reflectance and is coupled directly into a spectrometer (BWTek Inc., Newark, Del.). The second channel, which is dedicated to detection of fluorescence excited by the 633 nm PDT treatment beam, is long-pass filtered at 645 Tim (E645LP, Chroma Technology Corp., Rockingham, Vt.) before being directed into a second identical spectrometer. Both spectrometers are thermoelectrically (TE)-cooled 16-bit devices with 0.22 numerical aperture SMA inputs and detect 475-800 nm with 3 nm resolution. A LabVIEW (National Instruments, Austin, Tex.) program running on a laptop computer controls the system, obtains data, and performs real-time spectral analysis and display.

### Probe Specification

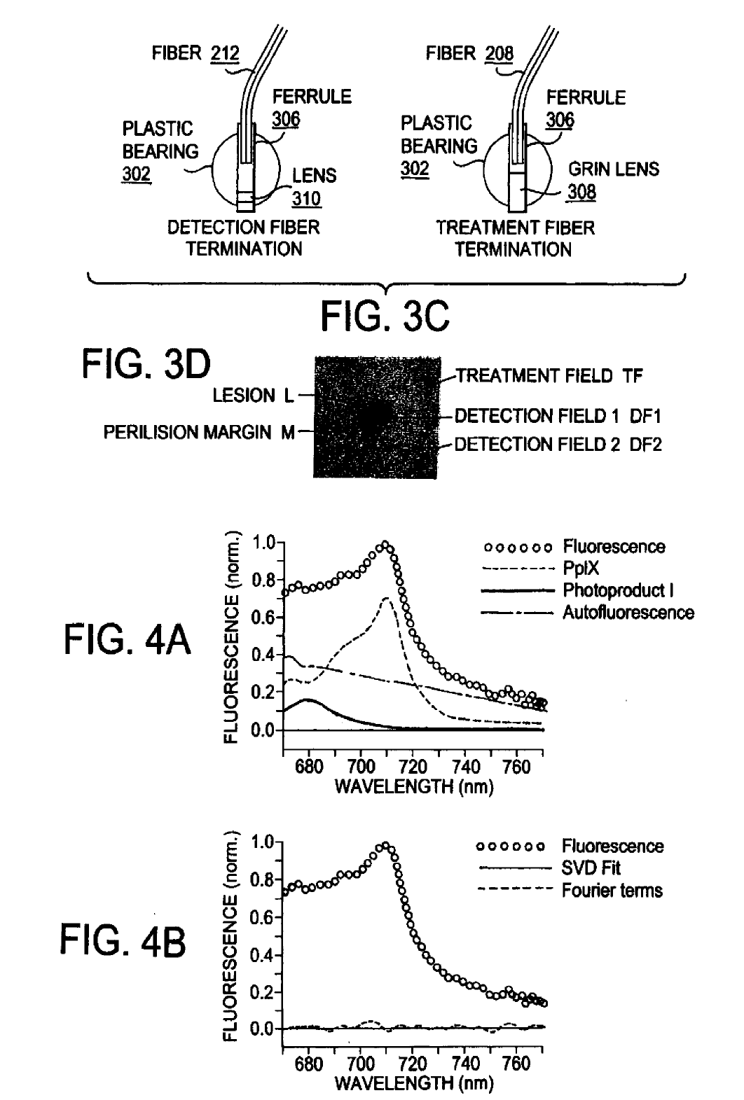

The fluorescence/reflectance probe was designed to measure tissue fluorescence and white light reflectance in identical geometries and is shown in FIGS. 3A-3D.

FIGS. 3A and 3B show a photograph an illustration of the off-surface fiber optic probe 210. The treatment fiber 208 and the detection fibers 212 terminate at plastic bearings 302. Legs 304 support the probe over an area to be treated. Light from the treatment fiber defines the treatment field TF, while light received by the detection fibers defines the detection fields, DF1, DF2.

FIG. 3C shows a single treatment fiber 208 terminated with a ferrule 306 and a gradient-index (GRIN) lens 308 and two detection fibers 212 terminated with ferrules 306 and lenses 310, housed in plastic bearings 312, which are used for beam aiming. FIG. 3D shows a representative placement of the treatment and detection fields TF, DF1, DF2 on a depiction of a BCC lesion L with detection from both the cancerous (DF1) and normal tissue (DF2) in the adjacent perilesion margin M.

The treatment beam is delivered through a 600 μm core, UV-VIS step-index fiber with a numerical aperture of 0.39 (Thor Labs Inc., Newton, N.J.). The distal end of this fiber is coupled to a 1.8 mm diameter, 0.25 pitch GRIN lens optimized for 630 nm (Newport Corp., Irvine Calif.) and housed within a 3 mm diameter aluminum tube. Similarly, the detection channels of the system consist of 365 μm core, UV-VIS step-index fibers with numerical apertures of 0.22 (Thor Labs) and terminated with 3 mm diameter, 6.7 mm focal length lenses (Edmund Optics Inc., Barrington, N.J.) in 4 mm diameter aluminum housing tubes, The probe housing consists of two flat plastic disks approximately 20 mm in diameter and ⅛ inch thick, milled with sockets for bearing placement. Three 10 mm diameter plastic bearings that have been milled with through holes are sandwiched between the disks to form a friction fit with the sockets, and the fibers are fixed into these bearings to allow continuous aiming adjustments before being locked in place with set screws prior to starting the therapy. Small gauge steel tubing is used to create three probe legs that are terminated with wire loops, allowing the probe to be fixed to the patient with medical tape. At 80 mm from the skin, the treatment field is 25 mm in diameter and uniform, and the two detection fields are 4 mm in diameter. The 25 mm illuminated area allows treatment of carcinomas up to 15 mm across with at least a 5 mm perilesional margin. Prior to treatment, the treatment and detection fields are properly oriented on the lesion by sending a trace beam generated with a modified laser pointer through the treatment and detection fibers. The treatment beam is centered on the lesion, and the first of the two detection fields is centered concentrically. The second detection field is placed just outside the primary lesion on normal tissue in the adjacent perilesion margin, as illustrated in FIG. 3D.

### System Control and Operation

A laptop computer (Thinkpad A51, IBM Corp., White Plains, N.Y.) running LabVIEW controls the system's operation. Communication with the two spectrometers is performed through USB ports, while control of the switches and the white light shutter is accomplished through the parallel port interfaced to a prototype board with amplification circuitry as described above. The LabVIEW interface contains fields for patient and lesion information as well as a spreadsheet in which the operator specifies details of the treatment, including time points for fluorescence and reflectance spectral acquisitions, spectroscopic integration times, and target treatment fluence and treatment fluence rates.

Prior to the start of treatment, the program initiates dark signal readings from each spectrometer. In patients requiring only local anesthetic, reflectance spectra are taken pre- and post-injection of anesthetic to explore possible changes to tissue optical properties caused by the intra-dermal 1% lidocaine (Xylocalne) injection. Prior to treatment, the instrument is configured with the white light shutter engaged and the second 2×1 fiber switch open to the white light source such that no light is directed through the delivery arm of the probe. Once the treatment is initiated, the instrument cycles through two intervals, treatment delivery and white light interrogation, with corresponding spectral data acquisitions. Fluorescence generated from the treatment beam is detected sequentially for a user-defined integration time from both the lesion and perilesion margin using the 2×2 switch. Fluorescence spectra are saved to file, analyzed in real time as described in detail below, and presented to the user in the LabVIEW interface. During the white light interval, the white light channel is opened in the second 2×1 switch, and light from the tungsten-halogen source is directed onto the treatment field. Reflectance from the two regions of interest is then detected sequentially with the second spectrometer, saved to file, and displayed for the user in the LabVIEW interface.

### Data Acquisition and Reduction

Fluorescence measurements have been made using the treatment fluence rate of 150 mW cm⁻². During treatment, these measurements were initially performed every 3 J cm² in order to sample the rapid initial decrease in sensitizer fluorescence. After 36 and 72 J cm⁻² the measurement intervals were increased to 4 and 6 J cm⁻² respectively, due to decreased signal strength and a more slowly varying bleaching curve. Spectrometer integration times for the fluorescence measurements were between 2 and 4 seconds. Qualitative inspection of preliminary results indicated only moderate therapy-induced changes to reflectance spectra, suggesting that infrequent data collection would be sufficient and would also serve to limit interruption of the 633 nm treatment delivery. Therefore, reflectance measurements were made immediately prior to the start of the treatment and then spaced by 18 J cm⁻² intervals. The spectrometer integration time for a reflectance measurement was 3 s.

Fluorescence: Prior to spectral analysis, the fluorescence data is corrected for instrumentation effects. Dark signals measured prior to treatment are subtracted from the raw data, and the spectrum is divided by a channel-specific sensitivity function, generated by measuring the instrument response to a NIST-traceable calibration lamp (LS-1-CAL, Ocean Optics, Dunedin, Fla.). In order to obtain intrinsic fluorescence, spectra must also be corrected for possible distortions introduced by tissue absorption and scattering. This can be accomplished using an empirical correction technique explored by Wu et al, in which the measured spectrum is divided by the white light reflectance spectrum measured in the same geometry and in the same spectral window. Though this method is easy to employ and provides a more rigorously corrected florescence spectrum, in practice we found that reflectance spectra between 655 and 800 nm were relatively flat, consistent throughout treatment, and subsequent corrections did not significantly affect data analysis. This correction is therefore omitted during the real-time data assessment of fluorescence and is only utilized for the more rigorous post measurement data analysis. In order to accurately analyze fluorescence spectra in real time, LabVIEW calls a MATLAB (Mathworks, Natick, Mass.) routine, which uses an SVD spectral fitting algorithm. The fluorescence basis spectra used in these analyses include those of PpIX, a fluorescent photoproduct of PpIX (photoproduct I), tissue autofluorescence, and fiber autofluorescence, all of which are NIST-traceably calibrated. The tissue autofluorescence spectrum used in the fitting is the normalized average of 18 spectra excited by 633 nm light measured from the forearms of two volunteers, and the fiber autofluorescence spectrum is the normalized average of 18 spectra measured by directing 633 nm light through the treatment channel of the instrument. The PpIX and photoproduct I basis spectra were obtained in previous work by Finlay et al. A 61-term Fourier series is also included in the spectral basis library to identify unaccounted-for contributions in the measured spectra and any possible autofluorescence changes induced by the therapy. A penalizing weight is given to this series, however, so that fitting preference is given to known bases. The total spectrum F(λ) is therefore represented by

$$F ( λ ) = {\sum }_{i = 1}^{4} {A}_{i} {w}_{1} {f}_{i} ( λ ) + {w}_{2} [ \begin{matrix}{B}_{0} + \\ {\sum }_{k = 1}^{61} {e}^{- \frac{k}{20}} \{ \begin{matrix}{B}_{k} sin ( \frac{π k ( λ - {λ}_{i} )}{{λ}_{f} - {λ}_{i}} ) + \\ {C}_{k} cos ( \frac{π k ( λ - {λ}_{i} )}{{λ}_{f} - {λ}_{i}} )\end{matrix} \}\end{matrix} ] ,$$

where w₁ and w₂ are the user-defined weights of the basis spectra, fᵢ(λ) are the spectra from known fluorophores, and Aᵢ, Bₖ, and Cₖ are the spectral amplitudes of the returned fit.

FIG. 4A shows a fluorescence spectrum measured from a BCC lesion after 3 J cm⁻² treatment and decomposed with SVD. The SVD fit shows appreciable contribution to the signal from PpIX, tissue and fiber autofluorescence, and photoproduct 1. As seen in FIG. 4B, a good fit to the data is achieved with known basis spectra and only a minimal contribution from the 61-term Fourier series. For clarity, the autofluorescence contribution shown is the sum of the tissue and fiber autofluorescence.

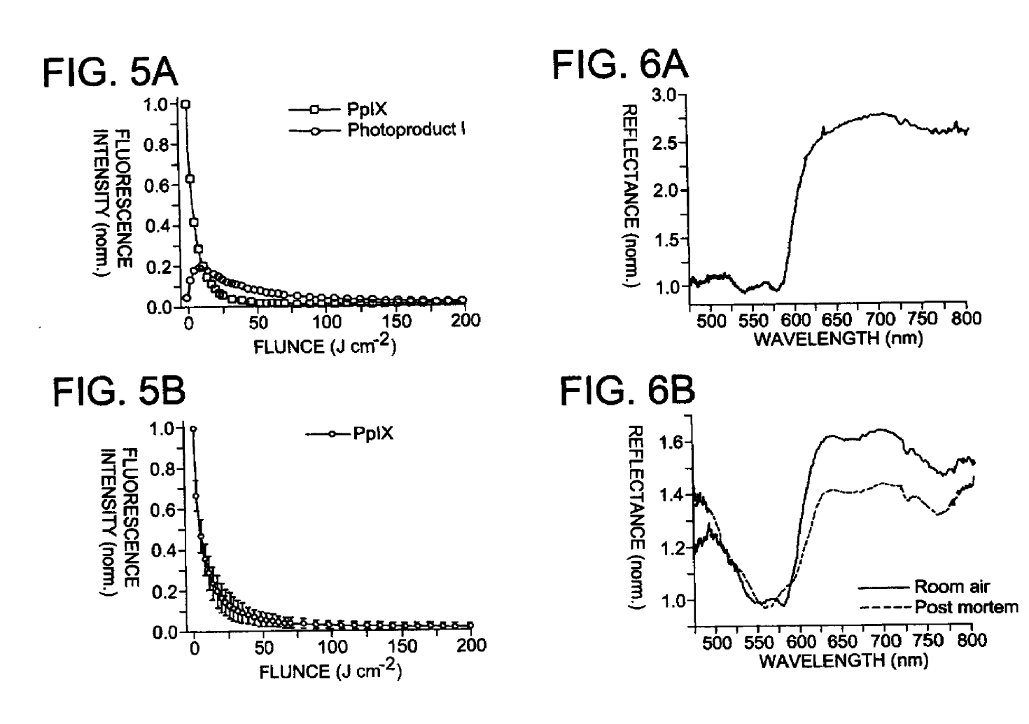

A representative fluorescence spectrum obtained from an ALA-sensitized basal cell carcinoma (BCC) lesion and analyzed with SVD is shown in FIGS. 4A and 4B. SVD analysis performed on each measured spectrum during the course of a treatment reports the relative amplitudes of the fluorophores represented in the fit. PpIX and photoproduct I contributions to the fluorescence from the lesion field throughout one PDT treatment delivered at 150 mW cm⁻² are shown in FIG. 5A.

FIG. 5A shows PpIX and photoproduct I contributions to fluorescence detected in the lesion field throughout a single PDT treatment of BCC of 200 J cm⁻² delivered at 150 mW cm⁻². The fluorescence intensity of the PpIX and photoproduct I plots are normalized to the initial PpIX fluorescence intensity detected from the lesion. Error bars represent errors in the SVD fit. FIG. 5B shows average PpIX fluorescence bleaching with the standard deviation for 7 lesions, representing 5 patients, treated for 200 J cm⁻² delivered at 150 mW cm⁻². As illustrated by the representative data in FIG. 5A, the PpIX bleaching curves for each of the individual lesions were smooth with fitting errors smaller than the data symbol. The larger uncertainties in the summary plot of FIG. 5B are therefore dominated by biological patient-to-patient variations. Initial photosensitizer bleaching was rapid, with 90% PpIX bleaching occurring at around 20 J cm⁻², representing only 10% of the 200 J cm⁻² prescribed fluence. The contribution to fluorescence from photoproduct I increases with delivered fluence until peaking at approximately 10 J cm⁻², after which it also undergoes photobleaching, although with a bleaching rate that is apparently slower than that of PpIX.

Reflectance: Reflectance spectra between 475 nm and 800 nm are collected separately for both fields. To correct white light reflectance spectra, dark signals measured prior to treatment are subtracted from the raw data and the spectrum is divided by a channel-specific sensitivity spectrum. A representative reflectance spectrum taken from the center of a BCC lesion is shown in FIG. 6A.

FIG. 6A shows a white-light reflectance spectrum measured from a BCC lesion after approximately 18 J cm⁻² treatment. FIG. 6B shows reflectance spectra from BALB/c mouse skin in vivo during normal respiration with room air and 10 minutes post mortem. The reflectance spectrum from the BCC lesion is qualitatively similar to that of the mouse during normal respiration with decreased reflectance at shorter wavelengths, possibly due to increased melanin in the skin, In the human reflectance data and reflectance data from mouse with normal respiration, the shape of the spectrum is qualitatively consistent with oxyhemoglobin. Similarly, in the reflectance data from the mouse post mortem, the qualitative shape of the spectrum is consistent with a shift to deoxyhemoglobin. Spectra have been normalized to the hemoglobin isosbestic point at 585 nm.

The spectrum shows the characteristics of oxyhemoglobin, and a small sharp peak at 633 nm that results from stray laser light in the clinic. FIG. 6B similarly shows reflectance spectra taken from normal BALB/c mouse skin in vivo during normal respiration of room air and at the same location 10 mm post mortem, between which qualitative differences are seen in significantly oxygenated and significantly deoxygenated blood, respectively. Throughout, the feature near 760 nm corresponds to an absorption band of deoxyhemoglobin. Rigorous analysis of the reflectance spectra acquired with the off-surface, overlapping source-detector probe geometry employed by this system is being developed.

### Preferred Embodiment for Feedback-Controlled PDT System

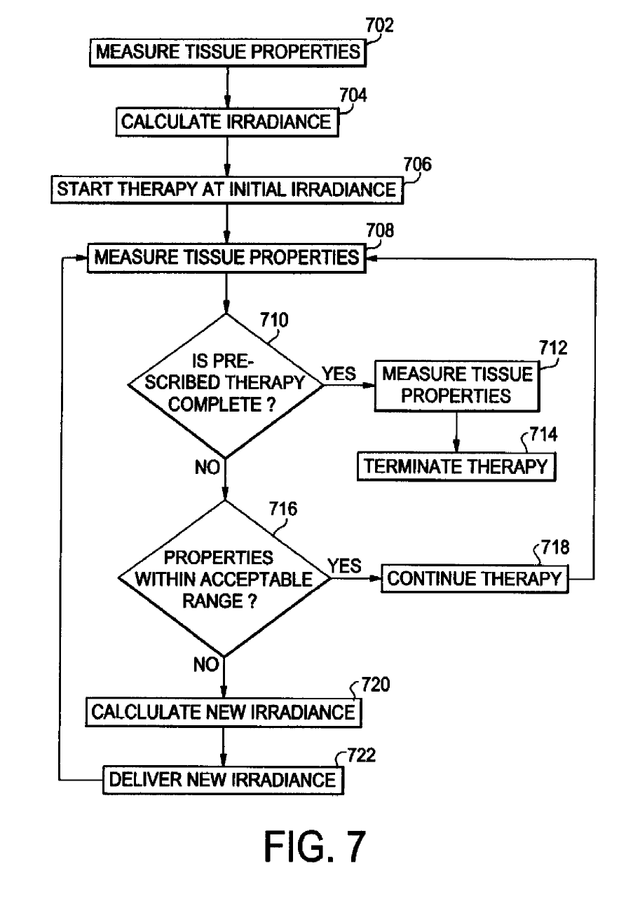

In this embodiment, the light delivery is feedback controlled to maintain a constant fluorescence photobleaching rate of the sensitizer. This serves to minimize pain during therapy while increasing efficiency of treatment delivery. Similarly, it could be used to maintain oxygen tension within the target region. The process is outlined in FIG. 7.

In step 702, tissue properties are measured. In step 704, the irradiance is calculated. In step 706, the therapy is started at the initial irradiance.

In step 708, the tissue properties are measured. It is determined in step 710 whether the prescribed therapy is complete. If so, the tissue properties are measured in step 712, and the therapy is terminated in step 716.

If the prescribed therapy is not complete, then in step 716 is determined whether the properties are within an acceptable range. If so, it is decided in step 718 to continue the therapy, and the process returns to step 708. Otherwise, a new irradiance is calculated in step 720 and delivered in step 722, and the process returns to step 708.

In this embodiment, the optical properties of the target region are measured using reflectance and/or fluorescence spectroscopy. The optical properties are used to determine a starting fluence rate based on oxygen availability, sensitizer levels, scattering, absorption, or other parameters within the target region. After the therapy is initiated at the starting fluence, the feedback into the system is the measured fluorescence intensity from the sensitizer within the target region. The difference between two sequential fluorescence intensity measurements divided by the time between the measurements approximates a sensitizer bleaching rate. The system takes the bleaching rate and calculates how much to increase the fluence rate in order to maintain that bleaching rate. The system continuously measures fluorescence and re-evaluates the fluence rate in order to keep the bleaching rate, the sensitizer-fluence product, or another quantity of interest constant. This bleaching rate can be established before the start of the therapy such that the delivery rate of PDT is below the pain threshold. Increasing the fluence rate as therapy proceeds, however, delivers the prescribed fluence quickly. If the therapy is delivered just below the pain threshold during the entire course of therapy, it will have been delivered in an optimal amount of time without causing pain. This concept is illustrated in FIG. 8, which shows a time course of therapy for PDT showing (a) non-feedback and (b) feedback-delivered PDT.

The photobleaching rate can, for example, be calculated by a computer controlling the system. The increase in fluence rate can subsequently be controlled by this computer varying the drive current to a laser, controlling a continuously adjustable neutral density filter, or some other means. The system described above could implement this technique by modest changes to the drive software and addition of a continuously adjustable neutral density filter in the path of the delivery laser, prior to being coupled into the optical switches.

It is an advantage of this embodiment that PDT is delivered with optimal efficiency, minimizing the patient's time in the clinic. It is also an advantage of this embodiment that PDT is delivered painlessly and therefore does not require anesthetics or conscious sedation. These advantages are conveyed in FIG. 8. The upper figure shows a typical sensitizer bleaching rate curve when the therapy is delivered at a single fluence rate. The bleaching rate initially starts high and then monotonically decreases during the therapy. Similarly, the lower figure depicts a sensitizer bleaching rate curve where the therapy is delivered with a constant bleaching rate and therefore a monotonically increasing fluence rate. In both cases, the therapy is delivered below the pain threshold, but a considerable time advantage is provided in the second case because the rate of light delivery is increased throughout the therapy in response to the measured loss of photosensitizer fluorescence.

Similarly, feedback control could be used to maintain oxygenation of the tissue, as reported by optically measured hemoglobin oxygen saturation, either by fluence rate control or treatment fractionation. Similarly, feedback could be used to determine an appropriate total fluence to the treatment region based on photosensitizer bleaching, generation of endogenous fluorophores, or other criteria.

While a preferred embodiment has been set forth above, those skilled in the art who have reviewed the present disclosure will readily appreciate that other embodiments can be realized within the scope of the invention. For example, numerical values are illustrative rather than limiting, as are recitations of specific suppliers or computer technologies. Therefore, the invention should be construed as limited only by the appended claims.

1. A method for delivering photodynamic therapy, said method comprising the steps of:
(a) irradiating a target region with a first irradiance;
(b) measuring a dosimetry parameter in said target region;
(c) calculating a clinically-preferred irradiance using said dosimetry parameter; and
(d) irradiating said target region at said clinically-preferred irradiance.

2. A method according to claim 1, wherein steps (c) and (d) are performed iteratively.

3. A method according to claim 2, wherein steps (c) and (d) are performed iteratively by:
(i) determining whether the therapy is complete;
(ii) if the therapy is not complete, determining whether the parameter is within an acceptable range;
(iii) if the parameter is within the acceptable range, continuing the therapy; and
(iv) if the parameter is not within the acceptable range, performing step (c) again.

4. A method according to claim 1, wherein said dosimetry parameter includes at least one of total fluorescence, rate of fluorescence photobleaching, change in fluorescence, fluence, fluence rate, blood oxygen saturation, absorption, change in scattering, temperature, photosensitizer concentration, blood perfusion, tissue oxygen, change in fluorophores, pain, and interstitial fluid pressure.

5. A device for delivering photodynamic therapy, said device comprising,
optics for providing radiation to a target region and for receiving radiation from the target region;
a sensor, in communication with the optics, for sensing the radiation received from the target region; and
a processor, in communication with the sensor, for measuring a dosimetry parameter in the target region and generating feedback control for the optics using the dosimetry parameter.

6. A device according to claim 5, wherein said dosimetry parameter includes at least one of total fluorescence, rate of fluorescence photobleaching, change in fluorescence, fluence, fluence rate, blood oxygen saturation, absorption, change in scattering, temperature, photosensitizer concentration, blood perfusion, tissue oxygen, change in fluorophores, pain, and interstitial fluid pressure.

7. A device according to claim 5, wherein the processor measures the dosimetry parameter and generates the feedback control iteratively.

8. A device according to claim 7, wherein the processor measures the dosimetry parameter and generates the feedback control iteratively by:
(i) determining whether the therapy is complete;
(ii) if the therapy is not complete, determining whether the parameter is within an acceptable range;
(iii) if the parameter is within the acceptable range, continuing the therapy; and
(iv) if the parameter is not within the acceptable range, generating the feedback control again.

9. A device according to claim 5, wherein the optics comprise a white light source and a treatment laser.

10. A device according to claim 9, wherein the optics further comprise:
a treatment fiber for causing the radiation to be incident on the target region; and
an optical switch for switching the radiation from the white light source or the treatment laser to the target region.

11. A device according to claim 10, wherein the optics further comprise a detection fiber for receiving the radiation to be received from the target region and for conveying the received radiation to the sensor.
